(19) United States
(12) Patent Application Publication
(10) Pub. No.: US 2007/0249893 A1
(43) Pub. Date: Oct. 25, 2007

(54) Cuff for a lumen

(76) Inventor: John Krumme, Woodside, CA (US)

Correspondence Address: MADSON & AUSTIN;GATEWAY TOWER WEST, SUITE 900, 15 WEST SOUTH TEMPLE, SALT LAKE CITY, UT, 84101, US

(21) Appl. No.: 11/411,221

(22) Filed: Apr. 25, 2006

(51) Int. Cl. A61F 2/02

(52) U.S. Cl. 600/31; 600/29

(57) ABSTRACT

A cuff for a lumen having at least one resilient strip with a base and first and second arms extending therefrom. Each of the first and second arms has a receiving face, the receiving faces being adapted to receive a lumen. At least one of the receiving faces has at least one inflatable bladder arranged to compress the lumen when inflated. An aperture is in fluid communication with the bladder such that the bladder can be inflated or deflated by passage of fluid through the aperture.

### BACKGROUND OF THE INVENTION

The present invention relates to a cuff for a lumen, in particular a urethra, and also a device for controlling urinary incontinence. More particularly, but not exclusively, the present invention relates to a urethral cuff comprising a U shaped support for controlling flow in a urethra, the cuff having a plurality of inflatable bladders.

Devices for compressing a urethra to control incontinence are known. Such devices typically comprise a tubular band which may be secured around the urethra. Controlled inflation or deflation of the tubular band on the outside of the urethra is used to control fluid flow in the urethra.

Such devices surround the urethra. This can restrict blood flow in the urethral wall. In implemented devices which are expected to function for long periods this can result in damage to the urethra. Circumferential devices also require surgical access fully around the urethra making surgical implantation difficult.

### SUMMARY OF THE INVENTION

The cuff according to the present invention seeks to overcome these problems. Accordingly, in a first aspect there is provided a cuff for a lumen comprising:

at least one resilient strip comprising a base and first and second arms extending therefrom, each of the first and second arms having a receiving face, the receiving faces being adapted to receive a lumen;

at least one of the receiving faces comprising at least one inflatable bladder arranged to compress the lumen when inflated; and

an aperture in fluid communication with the bladder such that the bladder can be inflated or deflated by passage of fluid through the aperture.

The cuff according to the invention grips the lumen only part way around its circumference and, by means of one or more bladders, at a plurality of separated points. This still has the effect of compressing the urethra so restricting fluid flow along it. However, in contrast to known devices the cuff according to the invention leaves undisturbed tissue between the bladders allowing blood flow so resulting in only minimal damage to the urethra.

A least one of the side arms can be planar.

At least one of the side arms can be curved.

Preferably, the resilient strip is U shaped.

Alternatively, the resilient strip can be V shaped.

Preferably, the base comprises at least one inflatable bladder.

At least one of the side arms can comprise a plurality of inflatable bladders. Preferably, the arm comprises a plurality of inflatable bladders.

Each bladder can be separated from the adjacent bladders by a non-inflatable portion. Preferably, the bladders are in fluid communication with each other by apertures through the non-inflatable portions.

Each bladder can comprise a ridge extending at least partially across the width of the strip.

Preferably, the urethral cuff comprises a plurality of strips, each comprising a plurality of bladders.

All of the bladders can be in fluid communication with the same aperture for simultaneous inflation or deflation of each bladder.

Alternatively, the bladders on at least one strip are adapted to be inflated or deflated independently of the remaining bladders.

The bladders on different strips of such a cuff can be inflated independently of each other. Different portions of the circumference of the lumen can therefore be contacted rather than the same part of the lumen being gripped each time the cuff is used. Because any one area of the urethra is compressed for less than full time damage to the urethra is reduced.

The cuff can further comprise a valve for controlling fluid flow through the aperture. Preferably, the cuff is adapted to receive a urethra.

In a further embodiment of the invention there is provided a device for controlling urinary incontinence comprising

at least one resilient strip comprising a base and first and second arms extending therefrom, each of the first and second arms having a receiving face, the receiving faces being adapted to receive a lumen;

at least one of the receiving faces comprising at least one inflatable bladder arranged to compress the lumen when inflated;

an aperture in fluid communication with the bladder such that the bladder can be inflated or deflated by passage of fluid through the aperture; and,

a fluid pump in fluid communication with the aperture for inflation of deflation of the bladders.

The device can comprise a reservoir in fluid communication with the pump such that the pump pumps fluid from the reservoir to the cuff. The reservoir can be elastic. The reservoir can comprise a septum.

### BRIEF DESCRIPTION OF THE DRAWINGS

The present invention will now be described by way of example only, and not in any limitative sense, with reference to the accompanying drawings in which

FIG. 1 shows a first embodiment of a cuff for a lumen according to the invention;

FIG. 2 shows a second embodiment of a cuff for a lumen according to the invention;

FIG. 3 shows a device for controlling urinary incontinence according to the invention comprising the cuff of FIG. 1 attached to a urethra; and

FIG. 4 shows a device for controlling urinary incontinence according to the invention including the cuff of FIG. 2.

### DETAILED DESCRIPTION OF THE INVENTION

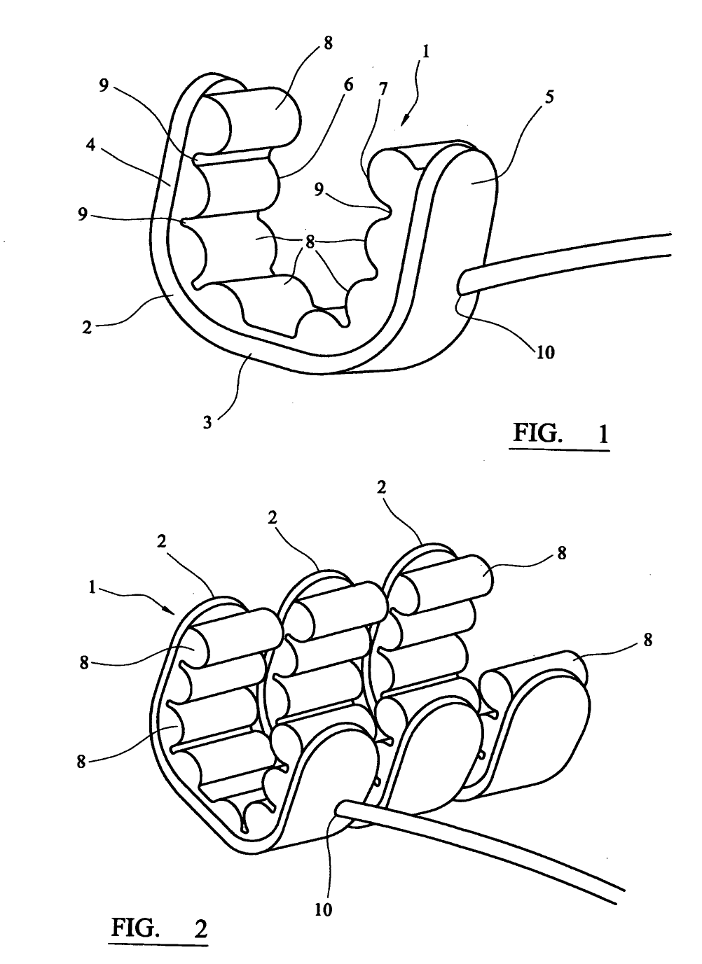

Shown in FIG. 1 is a cuff 1 for a lumen according to the invention. The cuff 1 comprises a U shaped resilient strip 2 having a base portion 3 and first and second side arms 4, 5 extending therefrom. On each of the inner facing sides 6, 7 of the side arms 4, 5 is a plurality of inflatable bladders 8. The base 3 also comprises a plurality of inflatable bladders 8. Each of the bladders 8 is shaped as a rib extending across the width of the strip 2. Each bladder 8 is separated from the adjacent bladder 8 by a non-inflatable portion 9 of the strip 2. Each bladder 8 is in fluid communication with its adjacent bladders 8 by apertures (not shown) through the non-inflatable portions 9 and is in fluid communication with an aperture 10 in the resilient strip. The bladders 8 may be inflated or deflated by the passage of fluid through the aperture 10. The cuff 1 is a biocompatible material such as a polymer. In this embodiment the cuff 1 is silicone. In an alternative embodiment the cuff 1 is urethane.

In use the lumen, in this case a urethra, is placed inside the U portion of the cuff 1 so gripping the urethra on three sides. The bladders 8 are inflated by the passage of fluid through the aperture 10 so squeezing the urethra and preventing fluid flow along the urethra. In order to allow fluid flow along the urethra the bladders 8 are deflated allowing the urethra to expand and the urethral lumen to expand.

The cuff 1 according to the invention is only in contact with the urethra at a number of spaced apart points. In contrast known devices contact the urethra around a significant portion, if not all of the circumference of the urethra. The cuff according to the invention therefore causes significantly less damage to the urethra in use than known devices and allows more fluid to flow along the urethral axis.

Shown in FIG. 2 is a further embodiment of a urethral cuff 1 according to the invention. The cuff 1 is similar to that of FIG. 1 except it comprises a plurality of resilient strips 2, each comprising a plurality of inflatable bladders 8. The bladders 8 on each strip 2 may be inflated and deflated independently of the bladders 8 on the adjacent strips 2.

In use the bladders 8 on one strip are inflated constricting the urethra so controlling fluid flow along the urethra. The next time the cuff 1 is used the bladders 8 on a different strip 2 of the cuff 1 are inflated so constricting the urethra in a different cyclical position along its length. This gripping of the urethra in a number of different places on a cyclical basis further reduces potential damage to the urethra. In this embodiment the cuff 1 has only one aperture 10 through which the bladders 8 on the strip 2 are inflated or deflated. In an alternative embodiment (not shown) each strip 2 has a separate aperture 10 through which the corresponding bladders 8 can be inflated or deflated.

In an alternative embodiment of the invention (not shown) all of the bladders 8 on the plurality of strips 2 can be inflated or deflated simultaneously.

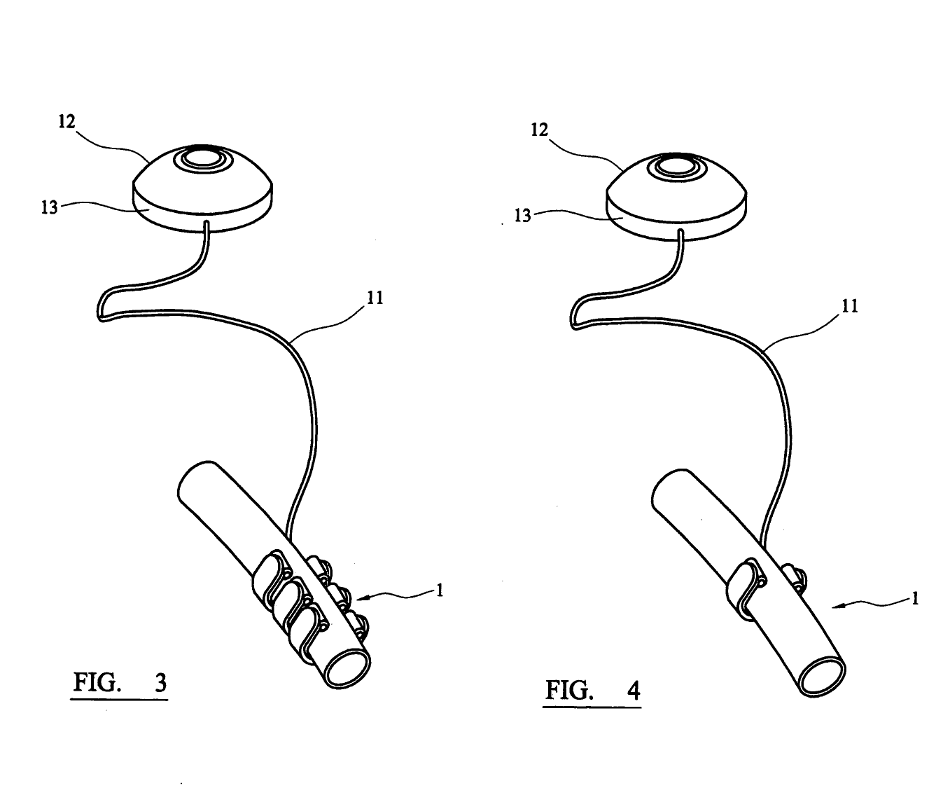

Shown in FIG. 3 is the cuff of FIG. 2 attached to a urethra. The arms of the cuff are connected together by a suture (not shown).

The cuff 1 is connected to a tube 11 which is in turn connected to a pump 12. The pump 12 pumps fluid from a fluid reservoir 13, through the tube 11 and though the aperture 10 to inflate the bladders 8. The bladders 8 are deflated by the reverse process. The cuff 1 has apertures for inflation or deflation of the bladders at each end (not shown) enabling the tube 11 to be connected to either end to aid implantation.

The cuff 1 of this embodiment includes a valve (not shown) for controlling fluid flow into and away from the cuff 1. The valve can be used to lock the cuff 1 in any desired state. For example at night the cuff 1 can be deflated so that no pressure is exerted on the urethra.

Shown in FIG. 4 is a device for controlling incontinence including the cuff of FIG. 1. This device is similar to that of FIG. 3 except it only includes one resilient strip

In a further embodiment of the invention (not shown) the pump pumps fluid from an elastic reservoir to the cuff 1. When the pump 12 is not powered the fluid is free to flow from the elastic reservoir, through the pump 12 to the cuff 1 so pressurising the bladders 8 to the same pressure as the reservoir. When the pump 12 is powered it can drive fluid either towards or away from the bladders 8. When the pump 12 is again unpowered the pressure in the bladders 8 returns to that in the reservoir. The static pressure in the elastic reservoir may be adjusted by means of a septum that allows the addition or subtraction of fluid volume to the reservoir.

In a further embodiment of the invention (not shown) the strip 2 is V shaped.

In a further embodiment of the invention (not shown) one of the arms 4, 5 comprises only one inflatable bladder 8.

In a further embodiment of the invention all of the bladders 8 are located on one arm 4, 5 of the strip 2.

1. A cuff for a lumen comprising:
at least one resilient strip comprising a base and first and second arms extending therefrom, each of the first and second arms having a receiving face, the receiving faces being adapted to receive a lumen;
at least one of the receiving faces comprising at least one inflatable bladder arranged to compress the lumen when inflated; and
an aperture in fluid communication with the bladder such that the bladder can be inflated or deflated by passage of fluid through the aperture.

2. A cuff as claimed in claim 1, wherein at least one of the side arms is planar.

3. A cuff as claimed in claim 1, wherein at least one of the side arms is curved.

4. A cuff as claimed in claim 1, wherein the resilient strip is U shaped.

5. A cuff as claimed in claim 1, wherein the resilient strip is V shaped.

6. A cuff as claimed in claim 1, wherein the base comprises at least one inflatable bladder.

7. A cuff as claimed in claim 1, wherein at least one of the side arms comprises a plurality of inflatable bladders.

8. A cuff as claimed in claim 7, wherein each arm comprises a plurality of inflatable bladders.

9. A cuff as claimed in claim 7, wherein each bladder is separated from the adjacent bladders by a non-inflatable portion.

10. A cuff as claimed in claim 9, wherein the bladders are in fluid communication with each other by apertures through the inflatable portions.

11. A cuff as claimed in claim 1, wherein the cuff comprises a plurality of bladders each in fluid communication with the apertures.

12. A cuff as claimed in claim 1, wherein the or each bladder comprises a ridge extending at least partially across the width of the strip.

13. A cuff as claimed in claim 1, comprising a plurality of resilient strips, each comprising a plurality of bladders.

14. A cuff as claimed in claim 13, wherein all the bladders are in fluid communication with the same aperture for simultaneous inflation or deflation of each bladder.

15. A cuff as claimed in claim 13, wherein the bladders on at least one strip are adapted to be inflated or deflated independently of the remaining bladders.

16. A cuff as claimed in claim 1, further comprising a valve for controlling fluid flow through the aperture.

17. A cuff as claimed in claim 1 where in the cuff is adapted to receive a urethra.

18. A device for controlling urinary incontinence comprising a cuff for a urethra as claimed in claim 1 and a fluid pump in fluid communication with the aperture for inflation of deflation of the bladders.

19. A device as claimed in claim 18, further comprising a reservoir in fluid communication with the pump such that the pump pumps fluid from the reservoir to the cuff.

20. A device as claimed in claim 19 wherein the reservoir is elastic.

21. A device as claimed in claim 19 wherein the reservoir comprises a septum.
